(19) United States
(12) Patent Application Publication
(10) Pub. No.: US 2023/0310070 A1
(43) Pub. Date: Oct. 5, 2023

(54) CATHETER

(71) Applicant: Japan Lifeline Co., Ltd., Tokyo (JP)

(72) Inventors: Kohei SAKAKI, Tokyo (JP); Makoto KATO, Tokyo (JP); Ryo ISHIDA, Tokyo (JP)

(21) Appl. No.: 18/186,014

(22) Filed: Mar. 17, 2023

(30) Foreign Application Priority Data
Mar. 29, 2022 (JP) 2022-052724

(51) Int. Cl. A61B 18/14; A61M 25/00

(52) U.S. Cl. CPC A61B 18/1492; A61M 25/0067

(57) ABSTRACT

Provided is a catheter in which a spline changes its shape into an intended shape. A catheter includes a shaft to be inserted into the body, a plurality of splines connected to the distal end side of the shaft, and a distal end member connected to the distal end side of the splines. The plurality of splines include a first spline and a second spline. The first spline and the second spline are located adjacent to each other and their motions are restrained by a first restraining member.

### TECHNICAL FIELD

The present disclosure relates to a catheter.

### BACKGROUND ART

A catheter is a type of medical devices to be inserted into the body for diagnosis or treatment. As an example, a catheter provided with a shaft and a basket electrode assembly coupled to a distal end of the shaft has been known (for example, see Patent Document 1). Here, the basket electrode assembly includes a plurality of splines. In addition, the basket electrode assembly is configured to change its shape from a contracted shape into an expanded shape by changing the shapes of the splines.

### CITATION LIST

### Patent Document

Patent Document 1: JP 2016-507349 T

### SUMMARY OF INVENTION

### Technical Problem

When the catheter described above is changed in shape with the splines in contact with a body tissue, the interval between the adjacent splines may become too narrow or too wide.

The present disclosure is made in view of the foregoing circumstances, and an object of the present disclosure is to provide a catheter that changes its shape with the adjacent splines held at appropriate intervals.

### Solution to Problem

A catheter according to the present disclosure includes a shaft to be inserted into a body, a plurality of splines connected to a distal end side of the shaft, and a distal end member connected to a distal end side of the splines. The plurality of splines include a first spline and a second spline. The first spline and the second spline are located adjacent to each other and motions of the first spline and the second spline are restrained by a first restraining member.

Any combination of the above components and conversions of representation of the present disclosure between a method, a device, a system, and the like are also effective as aspects of the present disclosure.

### Advantageous Effects of Invention

The catheter of the present disclosure can change its shape with splines adjacent to each other held at appropriate intervals.

### BRIEF DESCRIPTION OF DRAWINGS

FIG. 1 is a perspective view schematically illustrating an example of a state where a portion near a distal end of a catheter of the present disclosure is changed in shape.

FIG. 2 is a perspective view schematically illustrating another example of a state where the portion near the distal end of the catheter of the present disclosure is changed in shape.

FIG. 3 is a perspective view of members constituting the catheter illustrated in FIG. 1, from which splines are eliminated.

FIG. 4 is a perspective view of members constituting the catheter illustrated in FIG. 1, from which a distal end member is eliminated.

FIG. 5 is an enlarged view of an S portion illustrated in FIG. 4.

FIG. 6 is a side view schematically illustrating an example of the structure of the portion near the distal end of the catheter of the present disclosure.

FIG. 7 is a cross-sectional view taken along the line II-II in FIG. 6.

FIG. 8 is a cross-sectional view taken along the line III-III in FIG. 6.

FIG. 9 is a schematic diagram illustrating an example of the overall structure of the catheter of the present disclosure.

### DESCRIPTION OF EMBODIMENTS

The present disclosure will be described below based on preferred embodiments with reference to the drawings. The embodiments are illustrative and are not intended to limit the present disclosure. Not all features or combinations of the features described in the embodiments are essential to the present disclosure. The same or similar components, members, and processing operations illustrated in the drawings are denoted by the same reference numerals, and redundant descriptions are omitted as appropriate. The scales and shapes of the parts illustrated in each drawing are set for convenience to facilitate the explanation and should not be construed in a limited manner unless otherwise specified. When the terms “first”, “second”, and the like are used in the specification or claims, these terms do not mean any order or importance unless otherwise specified and are used for distinguishing a configuration from other configurations. Furthermore, some of the members that are not critical in describing the embodiments in the drawings are omitted in illustration.

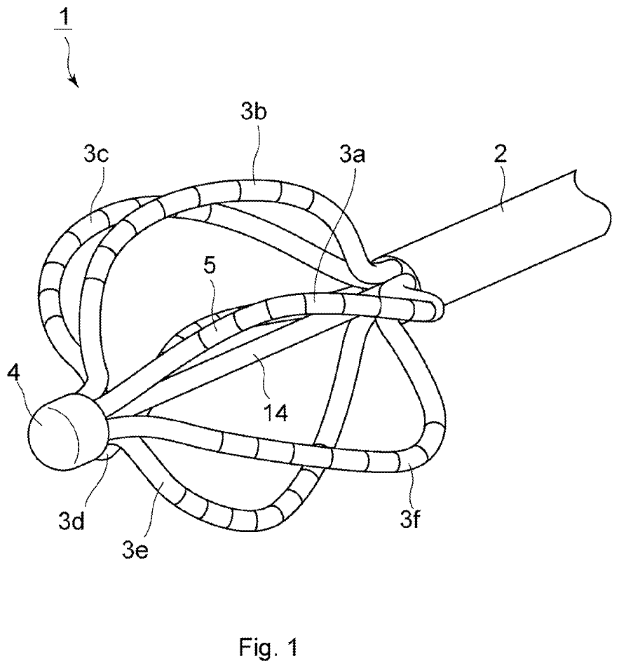

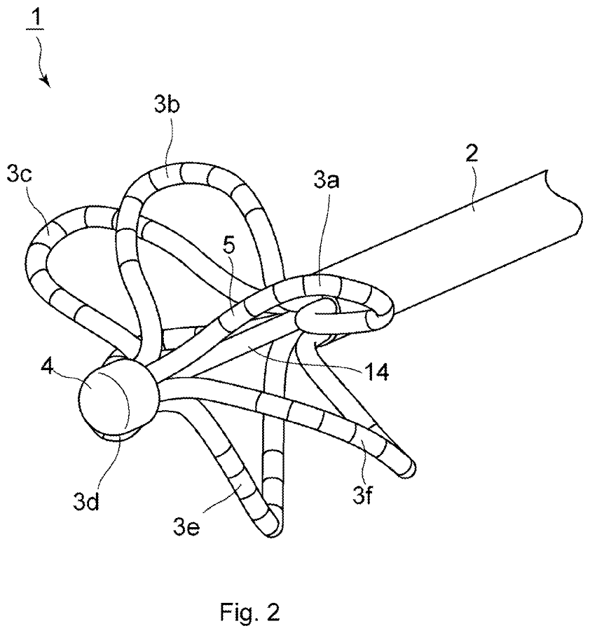

FIG. 1 is a perspective view schematically illustrating an example of a state where a portion near a distal end of a catheter 1 of the present disclosure is changed in shape. FIG. 2 is a perspective view schematically illustrating another example of a state where the portion near the distal end of the catheter 1 of the present disclosure is changed in shape. As illustrated in FIGS. 1 and 2, the catheter 1 of the present disclosure includes a shaft 2 to be inserted into the body, a plurality of splines 3 connected to a distal end side of the shaft 2, and a distal end member 4 connected to a distal end side of the splines 3.

The shaft 2 may be an elongated circular tubular member. The length of the shaft 2 is, for example, 800 mm to 1800 mm. The outer diameter of the shaft 2 is, for example, 2.0 mm to 5.0 mm. The material forming the shaft 2 may be a material having flexibility and biocompatibility. For example, the shaft 2 is formed of a known resin such as polyolefin or polyamide.

The spline 3 is a member that connects the shaft 2 and the distal end member 4. The spline 3 may be a circular tubular member like the shaft 2. The length of the spline 3 when extended in a linear manner is, for example, 20 mm to 50 mm. The outer diameter of the spline 3 is, for example, 0.5 mm to 2.0 mm. The material forming the spline 3 is any material having flexibility and biocompatibility. For example, the spline 3 is formed of a known resin such as polyolefin or polyamide, like the shaft 2.

The splines 3 include at least a first spline 3a and a second spline 3b that are adjacent to each other. FIGS. 1 and 2 illustrate an example in which the number of splines 3 is six. Specifically, an example is illustrated in which the first spline 3a is adjacent to the second spline 3b, the second spline 3b to a third spline 3c, the third spline 3c to a fourth spline 3d, the fourth spline 3d to a fifth spline 3e, the fifth spline 3e to a sixth spline 3f, and the sixth spline 3f to the first spline 3a. The splines 3 are disposed separated from each other in a plane orthogonal to a central axis of the shaft 2.

The splines 3 are connected to the shaft 2. For example, a portion (proximal end portion) including a proximal end of the spline 3 is inserted into the distal end side of the shaft 2. The proximal end portion of the spline 3 and the shaft 2 are joined to each other by a known joining method such as welding or bonding with an adhesive.

The spline 3 is configured to change its shape in accordance with a shape changing operation described below. Specifically, the shape of each spline 3 is changed between an unexpanded shape (a contracted shape) in which each spline 3 is not expanded along the central axis of the shaft 2 and an expanded shape (a developed shape) in which each spline 3 is expanded along the central axis of the shaft 2 from the unexpanded shape. Examples of the unexpanded shape may include a “petal shape” (see FIG. 2) as will hereinafter be described in detail. Meanwhile, examples of the expanded shape may include a “basket shape” (see FIG. 1) in which each spline 3 is expanded from the petal shape along the central axis of the shaft 2.

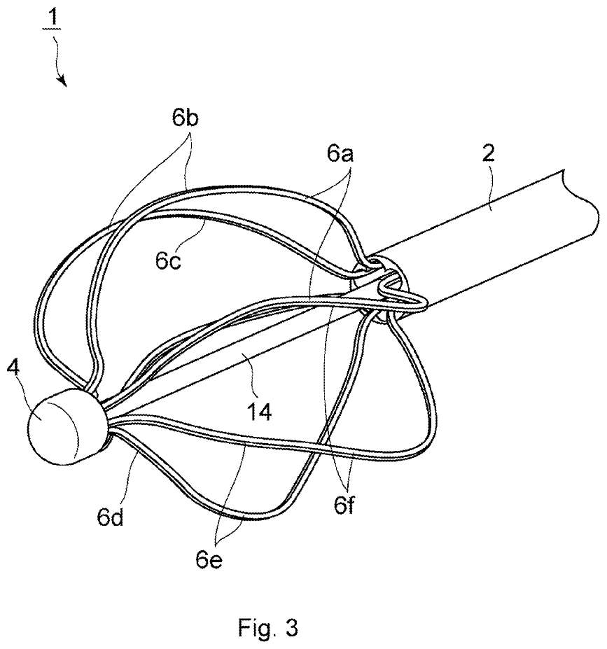

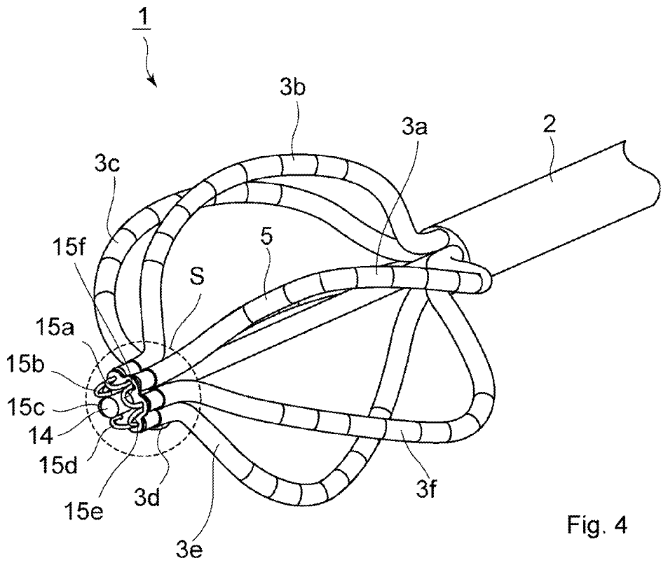

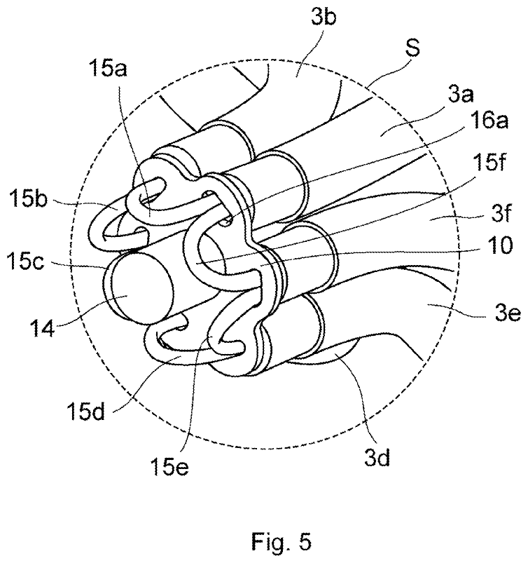

FIG. 3 is a perspective view of members constituting the catheter 1 illustrated in FIG. 1, from which the splines 3 are eliminated. FIG. 4 is a perspective view of members constituting the catheter 1 illustrated in FIG. 1, from which the distal end member 4 is eliminated. FIG. 5 is an enlarged view of the S portion illustrated in FIG. 4. As illustrated in FIGS. 3 to 5, motions of the first spline 3a and the second spline 3b are restrained by a first restraining member 6a. Here, that motions of the first spline 3a and the second spline 3b are restrained means that when the first spline 3a and the second spline 3b are changed in shape, the first spline 3a and the second spline 3b change their shapes while holding an appropriate interval therebetween. Note that the interval here is the shortest distance between the distal end side of the first spline 3a and the distal end side of the second spline 3b. Thus, even if the shape changing operation is performed in a state where the first spline 3a is in contact with a body tissue, the first spline 3a and the second spline 3b change its shape while holding an appropriate interval therebetween via the first restraining member 6a. Since satisfying such a configuration, the catheter 1 of the present disclosure can change its shape into a desired shape (unexpanded shape or expanded shape) such that the splines 3 adjacent to each other are held at appropriate intervals.

The first restraining member 6a may be of any shape, structure, and material as long as the member can restrain motions of the first spline 3a and the second spline 3b. The first restraining member 6a is wire, for example. The material forming the first restraining member 6a that is wire may be a shape-memory alloy such as a nickel-titanium alloy.

The first restraining member 6a may include a first end and a second end. At least a portion on the first end side of the first restraining member 6a is located within the first spline 3a, and at least a portion on the second end side of the first restraining member 6a may be located within the second spline 3b. The first restraining member 6a may include a protruding portion 15a protruding such that it is closer to the distal end of the catheter 1 than a distal end of the first spline 3a and a distal end of the second spline 3b are. Here, at least a portion on the first end side is a continuous portion having a certain length from the first end side of the protruding portion 15a in the first restraining member 6a. At least a portion of the second end side is a continuous portion having a certain length from the second end side of the protruding portion 15a in the first restraining member 6a.

The first end of the first restraining member 6a may be located closer to a proximal end of the catheter 1 than a proximal end of the first spline 3a is. Meanwhile, the second end of the first restraining member 6a may be located closer to the proximal end of the catheter 1 than a proximal end of the second spline 3b is. When such a configuration is satisfied, the first restraining member 6a serves as a framework that maintains the shapes of the first spline 3a and the second spline 3b.

In a case where the first restraining member 6a is wire, the protruding portion 15a may have a shape bent in a U-shape. When the first spline 3a or the second spline 3b is changed in shape, the first restraining member 6a is also changed in shape along with the spline. At this time, the stress generated by the change in shape is concentrated in the protruding portion 15a. When the protruding portion 15a has a shape bent in a U-shape, the stress described above can be effectively relieved, and even when changing in shape is repeatedly performed, the protruding portion 15a is less likely to break.

In the embodiment described above, the first spline 3a and the second spline 3b are described as an example; however, the same applies to the other splines 3. In other words, motions of the adjacent splines 3 may be restrained by one restraining member 6. Specifically, motions of the second spline 3b and the third spline 3c may be restrained by a second restraining member 6b. Motions of the third spline 3c and the fourth spline 3d may be restrained by a third restraining member 6c. Motions of the fourth spline 3d and the fifth spline 3e may be restrained by a fourth restraining member 6d. Motions of the fifth spline 3e and the sixth spline 3f may be restrained by a fifth restraining member 6e. Motions of the sixth spline 3f and the first spline 3a may be restrained by a sixth restraining member 6f.

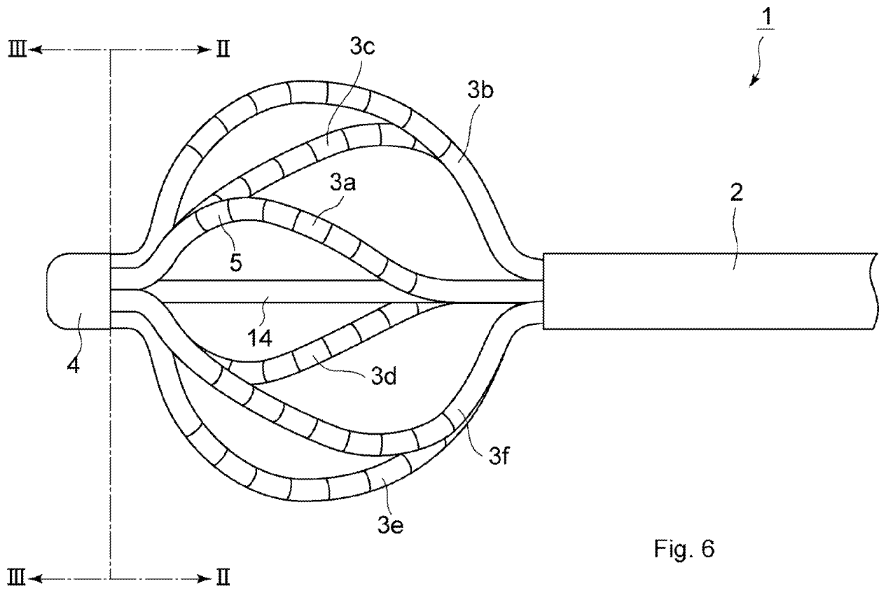

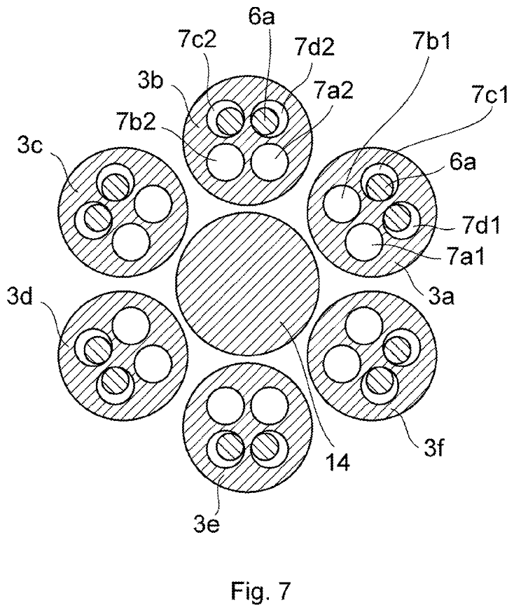

FIG. 6 is a side view schematically illustrating an example of the structure of the portion near the distal end of the catheter 1 of the present disclosure. FIG. 7 is a cross-sectional view taken along the line II-II in FIG. 6. As illustrated in FIG. 7, the first spline 3a may have a multi-lumen structure. Specifically, the first spline 3a includes four lumens 7 (7a1, 7b1, 7c1, 7d1) extending from the distal end to the proximal end along a central axis of the first spline 3a. Here, the lumens 7a1, 7b1 are located closer to the central axis of the shaft 2 than the lumens 7c1, 7d1 are. The lumens 7b1, 7c1 are located closer to the second spline 3b than the lumens 7a1, 7d1 are.

The second spline 3b may have a multi-lumen structure like the first spline 3a. Specifically, the second spline 3b includes four lumens 7 (7a2, 7b2, 7c2, 7d2) extending from the distal end to the proximal end along a central axis of the second spline 3b. Here, the lumens 7a2, 7b2 are located closer to the central axis of the shaft 2 than the lumens 7c2, 7d2 are. The lumens 7a2, 7d2 are located closer to the first spline 3a than the lumens 7c2, 7b2 are.

The first end side of the first restraining member 6a may be inserted into the lumen 7c1 of the first spline 3a. Meanwhile, the second end side of the first restraining member 6a may be inserted into the lumen 7d2 of the second spline 3b. As just described, the first restraining members 6a are inserted in the lumens 7c1, 7d2, located separated from the central axis side of the shaft 2 and closest to each other, of the lumens 7 in the first spline 3a and the second spline 3b, and thus the first spline 3a and the second spline 3b are held at a more appropriate interval.

In the embodiment described above, the first spline 3a, the second spline 3b, and the first restraining member 6a are described as an example; however, the same applies to the other splines 3 and the other restraining members 6.

Each spline 3 may include one or more electrodes 5 as illustrated in FIG. 6. The electrode 5 is a ring-shaped electrode for potential measurement or cauterization. The electrodes 5 included in the same spline 3 are disposed separated from each other along the longitudinal direction of the spline 3. In this case, the intervals between the adjacent electrodes 5 may be uniform or may be different. The number of electrodes 5 included in each of the splines 3 may be the same or may be different.

The electrode 5 is formed of a material having conductivity. For example, the electrode 5 is formed of a metal which has a good electrical conductivity, such as aluminum (Al), copper (Cu), stainless steel, gold (Au), or platinum (Pt). The length of the electrode 5 is, for example, 0.5 mm to 2.0 mm. The outer diameter of the electrode 5 is equivalent to the outer diameter of the spline 3, and is, for example, 0.5 mm to 1.6 mm.

A conductive wire is individually and electrically connected to each of the electrodes 5. The conductive wire passes through the spline 3, the shaft 2, and a handle 8 described below to be connected via the handle 8 to an external power supply device. For example, the conductive wires connected to the respective electrodes 5 of the first spline 3a pass through the lumen 7a1 or the lumen 7b1 of the first spline 3a while being electrically isolated from each other. Note that the same applies to the splines 3 other than the first spline 3a.

As illustrated in FIGS. 2 and 4, the distal end member 4 may cover a portion (distal end portion) including the distal end of the first spline 3a, a portion (distal end portion) including the distal end of the second spline 3b, and the protruding portion 15a of the first restraining member 6a. In other words, the distal end portion of the first spline 3a, the distal end portion of the second spline 3b, and the protruding portion 15a of the first restraining member 6a may be covered by the distal end member 4. The distal end member 4 may have any shape, and has a cap shape, for example. The distal end member 4 may be formed of any material, and is formed of a known resin such as nylon, nylon elastomer, or polycarbonate, or a known metal such as stainless steel. Note that the distal end member 4 may cover all of the distal end portions of the splines 3 and the protruding portions 15 of the restraining members 6.

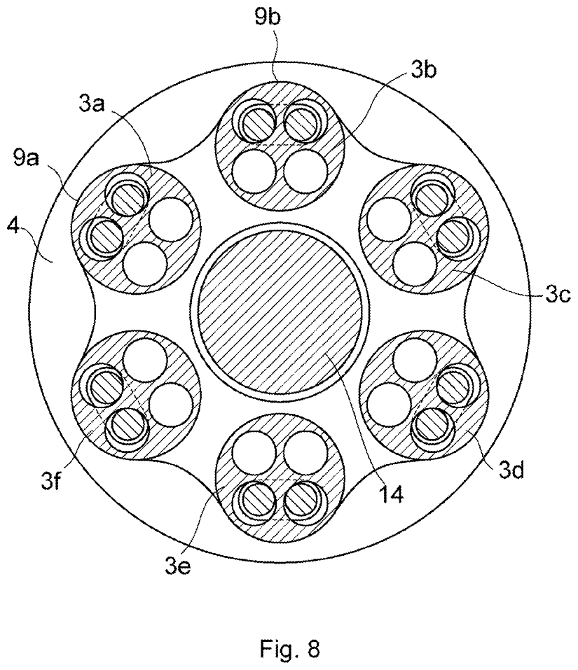

FIG. 8 is a cross-sectional view taken along the line III-III in FIG. 6. As illustrated in FIG. 8, in plan view of a lower surface of the distal end member 4, a wall portion forming an inner surface of the distal end member 4 may have a first recess portion 9a and a second recess portion 9b that are located adjacent to each other. At least a portion of the distal end portion of the first spline 3a may be located in the first recess portion 9a, and at least a portion of the distal end portion of the second spline 3b may be located in the second recess portion 9b. Here, that at least a portion of the distal end portion of the first spline 3a is located in the first recess portion 9a means that in plan view of the lower surface of the distal end member 4, a region of the first recess portion 9a includes a portion overlapped by the first spline 3a. Meanwhile, that at least a portion of the distal end portion of the second spline 3b is located in the second recess portion 9b means that in plan view of the lower surface of the distal end member 4, a region of the second recess portion 9a includes a portion overlapped by the second spline 3b. In a case where such a configuration is satisfied, when the first spline 3a and the second spline 3b are changed in shape, the distal end portion of the first spline 3a and the distal end portion of the second spline 3b can be prevented from becoming misaligned, by the first recess portion 9a and the second recess portion 9b.

The shape of the first recess portion 9a and the second recess portion 9b is, for example, a semicircle in plan view of the lower surface of the distal end member 4. The wall portion forming the first recess portion 9a may be in contact with but need not be in contact with the first spline 3a. Similarly, the wall portion forming the second recess portion 9b may be in contact with but need not be in contact with the second spline 3b. Note that FIG. 8 illustrates an example in which the wall portion forming the inner surface of the distal end member 4 has six recess portions 9. As just described, the wall portion forming the inner surface of the distal end member 4 may have the recess portions 9 corresponding to the respective splines 3.

The interior of the distal end member 4 may be filled with an adhesive. When such a configuration is satisfied, the first spline 3a, the second spline 3b, and the first restraining member 6a are firmly joined to each other by the adhesive. Therefore, the first spline 3a and the second spline 3b are held at a more appropriate interval.

As illustrated in FIG. 5, the distal end member 4 may include a bundling member 10 that bundles the protruding portion 15a of the first restraining member 6a. Here, that bundling the protruding portion 15a means that bundling the protruding portion 15a such that the protruding portion 15a does not move in a planar direction orthogonal to the central axis of the shaft 2. When such a configuration is satisfied, the twisting of the first restraining member 6a is suppressed by the bundling member 10, and thus the first spline 3a and the second spline 3b can be changed in shape via the first restraining member 6a effectively in conjunction with each other. Note that the bundling member 10 may bundle the protruding portions 15 of all of the restraining members 6.

The bundling member 10 may be of any shape, structure, and material as long as the bundling member 10 can bundle the protruding portion 15a. For example, the shape of the bundling member 10 is a plate including a first surface (top surface) and a second surface (lower surface) that is the back surface of the first surface. The second surface faces a distal end surface of the first spline 3a and a distal end surface of the second spline 3b. The bundling member 10 is provided with a through-hole 16a penetrating from the first surface to the second surface. The protruding portion 15a of the first restraining member 6a is inserted into the through-hole 16a. A third surface (side surface) that connects the first surface and the second surface of the bundling member 10 may have any shape that corresponds to the wall portion forming the inner surface of the distal end member 4. Specifically, the third surface may have a shape that includes protrusion portions corresponding to the respective recess portions 9 of the distal end member 4. The material forming the bundling member 10 may be a material having high rigidity. For example, the bundling member 10 is formed of a known metal such as stainless steel. Note that the bundling member 10 may be provided with through-holes 16 corresponding to the protruding portions 15 of the respective restraining members 6.

The through-hole 16a may have any shape as long as the protruding portion 15a of the first restraining member 6a can be inserted into the through-hole 16a. For example, the shape of the through-hole 16a is an elliptical shape in plan view seen from the first surface side of the bundling member 10. Note that the same may apply to the other through-holes 16.

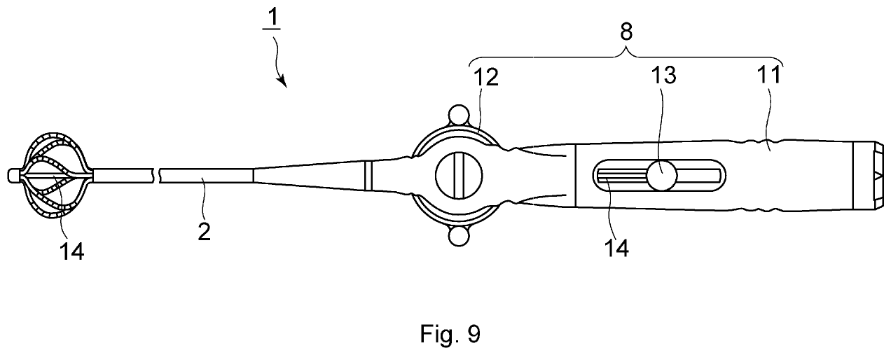

FIG. 9 is a schematic diagram illustrating an example of the overall structure of the catheter 1 of the present disclosure. As illustrated in FIG. 9, the catheter 1 may be provided with the handle 8 connected to the proximal end side of the shaft 2. The handle 8 is a portion that an operator (physician) grips (holds) when using the catheter 1. The handle 8 may include a handle body 11 mounted on the proximal end side of the shaft 2, a rotational operation portion 12, and a slide member 13.

The handle body 11 corresponds to a portion that is actually held by the operator. The handle body 11 may have any shape. The handle body 11 has a shape extending along the central axis of the shaft 2, for example. In addition, the handle body 11 is formed of, for example, a known resin such as polycarbonate, polyacetal, or ABS.

The rotational operation portion 12 is a portion that is operated (rotated) when a portion near the distal end of the shaft 2 is bent (warped) in both directions. Proximal ends of a pair of pull wires are fixed to the rotational operation portion 12 in the handle body 11. Distal ends of the pair of pull wires pass through the handle body 11 and the shaft 2 to be fixed to the distal end side of the shaft 2. Thus, when the rotational operation portion 12 is operated (rotated), the pull wires are pulled toward the proximal end side, and the distal end side of the shaft 2 to which the distal ends of the pull wires are fixed is bent (warped).

The slide member 13 is a portion that is changed in shape (slid) by the operator at the time of changing the shape of the spline 3 between the unexpanded shape (contracted shape) and the expanded shape (developed shape) that are described above. The slide member 13 is slidable along the central axis of the shaft 2 in the handle body 11.

A proximal end of a shape changing member 14 is fixed to the slide member 13. A distal end of the shape changing member 14 passes through the shaft 2 from the inside of the handle body 11 to be fixed to the inside of the distal end member 4. The slide member 13 is movable to any position in the handle body 11 along the central axis of the shaft 2. Therefore, in accordance with the position of the slide member 13, the shape of the spline 3 can be changed to the aforementioned unexpanded shape (contracted shape), the aforementioned expanded shape (developed shape), or any intermediate shape between the unexpanded shape and the expanded shape.

The shape changing member 14 may be of any shape, structure, and material as long as the shape changing member 14 is an elongated member. For example, the shape changing member 14 is wire thicker than the restraining member 6.

The embodiment of the present disclosure has been described in detail. The embodiment described above is merely a specific example for carrying out the present disclosure. The content of the embodiment is not intended to limit the technical scope of the present disclosure. Many design changes such as changes, additions, and deletions of components can be made in the scope that does not depart from the spirit of the present disclosure specified in the claims. A new embodiment with design changes has effects of combined embodiments and variations thereof. Any combination of components included in each embodiment is also effective as an aspect of the present disclosure. Hatching in sections of the drawings does not limit the material of a hatched object.

### REFERENCE SIGNS LIST

1: Catheter
2: Shaft
3, 3a, 3b, 3c, 3d, 3e, 3f: Spline
4: Distal end member
5: Electrode
6, 6a, 6b, 6c, 6d, 6e, 6f: Restraining member
7, 7a1, 7b1, 7c1, 7d1, 7a2, 7b2, 7c2, 7d2: Lumen
8: Handle
9, 9a, 9b: Recess portion
10: Bundling member
11: Handle body
12: Rotational operation portion
13: Slide member
14: Shape changing member
15, 15a, 15b, 15c, 15d, 15e, 15f: Protruding portion
16, 16a: Through-hole

1. A catheter comprising:
a shaft to be inserted into a body;
a plurality of splines connected to a distal end side of the shaft; and
a distal end member connected to a distal end side of the splines, wherein
the plurality of splines include a first spline and a second spline, and
the first spline and the second spline are located adjacent to each other and motions of the first spline and the second spline are restrained by a first restraining member.

2. The catheter according to claim 1, wherein
the first restraining member includes a first end and a second end,
at least a portion on the first end side of the first restraining member is located within the first spline,
at least a portion on the second end side of the first restraining member is located within the second spline, and
the first restraining member includes a protruding portion protruding such that the protruding portion is closer to a distal end of the catheter than a distal end of the first spline and a distal end of the second spline are.

3. The catheter according to claim 2, wherein the distal end member covers a distal end portion of the first spline, a distal end portion of the second spline, and the protruding portion of the first restraining member.

4. The catheter according to claim 3, wherein
in plan view of a lower surface of the distal end member, a wall portion forming an inner surface of the distal end member includes a first recess portion and a second recess that are located adjacent to each other,
at least a portion of the distal end portion of the first spline is located in the first recess portion, and
at least a portion of the distal end portion of the second spline is located in the second recess portion.

5. The catheter according to claim 4, wherein an interior of the distal end member is filled with an adhesive.

6. The catheter according to claim 3, wherein the distal end member includes a bundling member bundling the protruding portion of the first restraining member.

7. The catheter according to claim 6, wherein
the bundling member has a plate shape including a first surface and a second surface that is a back surface of the first surface,
the second surface faces a distal end surface of the first spline and a distal end surface of the second spline, and
the bundling member is provided with a through-hole which penetrates from the first surface to the second surface and into which the protruding portion of the first restraining member is inserted.

8. The catheter according to claim 1, wherein the first restraining member is wire.

9. The catheter according to claim 1, wherein the first restraining member is provided separately from the distal end member and the shaft.

10. A catheter comprising:
a shaft to be inserted into a body;
a plurality of splines connected to a distal end side of the shaft; and
a distal end member connected to a distal end side of the splines, wherein
motions of the splines adjacent to each other are restrained by one restraining member.
